(19) United States
(12) Patent Application Publication
(10) Pub. No.: US 2024/0341370 A1
(43) Pub. Date: Oct. 17, 2024

(54) Fecal Incontinence Undergarment

(71) Applicant: Marc Anthony Morales, Yonkers, NY (US)

(72) Inventor: Marc Anthony Morales, Yonkers, NY (US)

(21) Appl. No.: 18/300,244

(22) Filed: Apr. 13, 2023

(51) Int. Cl. A41B 9/02; A41B 9/00

(52) U.S. Cl. CPC A41B 9/023; A61F 5/4404

(57) ABSTRACT

An undergarment for individuals suffering from fecal incontinence with an aperture for access to the genitals and secure coverage over the anus that is intended to be worn during activities such as sexual activity. The undergarment is structured to contain stool should an involuntary bowel movement occur during such activities. The material of the garment stretches and conforms to the user's body and can also be used with a removable insert to assist with the containment of stool.

### BACKGROUND OF THE INVENTION

### Field of the Invention

This disclosure relates to incontinence undergarments and is specifically related to incontinence devices with access to the genitals for facilitating a number of activities, including sexual activity.

### Discussion of the Prior Art

Many adults experience fecal incontinence, which causes an involuntary loss of bowel contents. While this is commonly thought of as a condition found in those of advanced age, many younger individuals also experience incontinence as a result of disease, cancer, organ failure, physical injury, or other disorders. Although the prior art teaches adult incontinence undergarments that function by containing fecal matter within the undergarment, these products are generally bulky, uncomfortable, and unattractive. Additionally, these undergarments do not permit certain activities while being worn, such as sexual activity, as the genitals are covered by the undergarment.

The prior art also teaches undergarments that are crotchless and can be worn during sexual activity. The prior art also teaches undergarments designed for those that suffer from urinary incontinence. However, these undergarments are not designed for users who suffer from fecal incontinence and provide no mechanism to contain an involuntary bowel movement. Healthcare professionals and individuals alternatively attempt to treat incontinence in adults with medication by strictly controlling diet, timing sexual activity in accordance with their bowel movements, or using enemas. While these methods do not rely on a physical undergarment to be worn, they are unsatisfactory for many users. Individuals with medical conditions that cause fecal incontinence may wish to avoid additional medications due to potential complications with their condition, interactions with other medications, or simply personal preference. Only participating in sexual activity at specific times or after taking an enema can be far too restrictive and onerous for many people. Moreover, the effectiveness of these methods can vary from person to person, leaving a significant risk of an involuntary bowel movement at any time, which would disrupt sexual activity.

Such an event occurring while the individual is not wearing anything to contain fecal matter, as well as the associated odors, would likely cause embarrassment to the user and elicit a negative reaction from their partner. These incidents, or even the threat of such incidents, can reduce sexual desire and interfere with sexual performance. This risk is exacerbated by the body's own physical response during sex, which relaxes certain muscles making it more likely an involuntary bowel movement may occur. It is highly desirable to minimize such risk during sexual activity.

Thus, the need for a better solution for individuals with incontinence to confidently participate in sexual activities remains unaddressed. It would be desirable to provide a solution that permits an incontinent individual to participate in sexual activities with a degree of assurance that if an involuntary bowel movement were to occur, the fecal matter and associated odors would be contained, allowing the sexual activity to continue or allowing the user to excuse himself and easily dispose of any feces in the toilet. Such a solution would permit individuals suffering from incontinence to confidently participate in sexual activity without having to rely on a strict diet, medication, or timing regimens.

### BRIEF DESCRIPTION OF THE INVENTION

The present invention addresses the problems identified above by providing an incontinence undergarment that a user can wear during sexual activity, which traps fecal matter released by the user and its associated odors while permitting free access to the genitals. The undergarment is worn by the user in a manner similar to how underpants are worn. The undergarment includes a barrier adjacent to the anus of the user. The anterior face of the undergarment provides for an opening that permits access to the user's genitals. For males, the opening is sufficient for a user's erect penis and testicles to extend through. For females, the opening provides unimpeded access to the user's labia minora, clitoris, and vaginal opening. The undergarment provides a tight fit around this opening which prevents stool from escaping beyond the interior of the undergarment should the guard fail to contain it. The undergarment permits users to freely engage in sexual intercourse or other activities involving direct contact with the genitals, while the undergarment is worn by one or both users. The undergarment is made up of material that provides durability and comfort for the user. It maintains an appealing visual appearance that is suitable for sexual activity.

In accordance with one embodiment of the invention, the invention comprises an incontinence undergarment with an anterior opening aligned with the external genitals of a male, including the penis and testicles.

In accordance with another embodiment of the invention, the invention comprises an incontinence undergarment with an anterior opening aligned with the external genitals of a female.

In accordance with another embodiment of the invention, the invention comprises an incontinence undergarment with a removable plastic insert adjacent to the anus that can be cleaned after use allowing the undergarment to be reused.

In accordance with another embodiment of the invention, the invention comprises an incontinence undergarment with a disposable insert adjacent to the anus, which can be disposed of after use.

Various elements of the invention may be used alone or in combination. Numerous aspects of the present invention are presented herein. There are other aspects, features, and advantages of the present invention that will become more readily apparent from the attached drawings and the detailed description of the preferred embodiments which follow.

An incontinence undergarment for sexual activity is illustrated in various configurations in the figures.

### BRIEF DESCRIPTION OF THE DRAWINGS

FIG. 1 is a representative example of an anterior view of the fecal incontinence undergarment depicting the opening for access to the user's genitals.

FIG. 2 is a representative example of a posterior view of a male user wearing the incontinence undergarment with the removable guard.

FIG. 3 is a representative example of a side view of a male user wearing the incontinence undergarment.

FIG. 4 is a representative example of a side view of a male user wearing the incontinence undergarment with the guard.

FIG. 5 is a representative example of an anterior view of an embodiment of the incontinence garment with larger openings.

FIG. 6 is a representative example of a top view of the insert.

FIG. 7 is a representative example of a bottom view of the plastic insert.

### DETAILED DESCRIPTION OF THE INVENTION

The incontinence undergarment provides a method of minimizing undesired consequences of involuntary bowel movements during sexual activity. It can also be used by incontinent individuals during other activities where access to the genitals is required, such as urination while standing. When worn as a second layer under another garment, the benefits of the invention can also be taken advantage of during other activities such as sports, including swimming, or to prevent a male's genitals from sticking to other parts of the skin due to perspiration. The undergarment is form-fitting, attractive, and comfortable, creating a minimum level of intrusion or distraction during sexual activity. It comes in different configurations for males and females to account for male and female anatomy, such as the location of the genitals and the anus. While the description of the typical user is referred to as male, that should not be construed to limit any aspect of the invention to males only unless explicitly stated. The undergarment can also be manufactured in a variety of colors to present a pleasing visual aesthetic for the user and his partner or to blend seamlessly with the user's particular skin tone. The terms stool and feces are used interchangeably throughout this description.

The incontinence undergarment comprises an elastic waistband commonly found similar to the waistband of men's underwear and is also intended to be worn in a manner similar to underwear. The material of the undergarment comprises fabric that is soft, breathable, moisture-wicking, and contains a high degree of elasticity. When worn, the undergarment will stretch and conform to the user's body. An aperture on the anterior face of the undergarment provides direct access to the user's genitals. The undergarment further comprises a nonpermeable removable insert, or guard, between the user and the inside face of the posterior of the undergarment. The guard acts as a barrier between the user and the posterior side of the undergarment. The guard is held in position against the user's body over the anus by the elastic properties of the material of the undergarment. Additional fasteners may also be used to further secure the guard.

The genital aperture on the anterior face of the undergarment permits the user to engage in sexual activity involving the genitals while wearing the incontinence undergarment. In many individuals that suffer from incontinence, bowel control is lost due to control of the sphincter muscles. If a loss of bowel control occurs during sexual activity, stool released by the user will come into contact with the guard. The bowel movement will then be halted as the guard will contain any stool released within the intergluteal cleft by the guard, the user's buttocks, and the user's anus. The user may then remove the undergarment and guard, allowing stool previously trapped within the intergluteal cleft to be disposed of in the toilet, and wash any remaining stool residue from the guard using soap and water. As the stool will be contained within the intergluteal cleft, the guard will have prevented stool from contaminating other parts of the undergarment or areas outside of the undergarment. The nonpermeable attributes of the guard will also contain the unpleasant odors from the stool to some degree. The guard can then be reinserted into the undergarment and immediately reused.

The incontinence undergarment may be worn all day under a pair of the user's regular underwear, but users may also put the undergarment on just prior to a sexual encounter. When referencing the undergarment, the terms garment and undergarment are used interchangeably. The conforming fit of the undergarment is aesthetically pleasing, which minimizes distraction from the sexual encounter, and in some cases, may enhance it. The undergarment can be produced in a variety of colors, including skin tones that blend with a user's natural skin as well as high-contrast colors such as black or white.

The incontinence undergarment comprises four apertures. Orientations referenced are with respect to an individual standing upright. References to the parts of the human anatomy to describe the structure of the invention refer to the external portions of the anatomy. For example, references to the penis when describing the structure of the invention refer to the body or external portion of the penis.

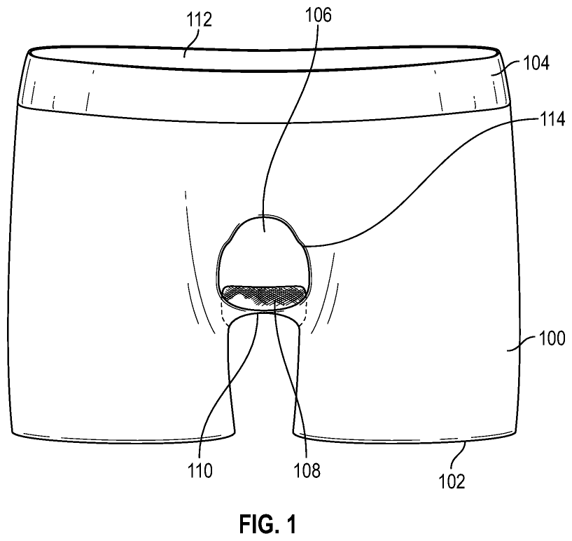

A left-leg aperture and a right-leg aperture are provided for the user to downwardly insert each of his legs. The larger torso aperture provides a vertical opening and is located at the top of the undergarment for the user's torso. As depicted in FIG. 1, the anterior of the undergarment has a genital aperture 106 which provides a horizontal opening that is positioned to encompass the user's genitals when worn. For a male user, this aperture will permit a user's testicles and penis, either in both an erect or flaccid state, to extend through the opening 106. For a female user, this aperture will provide access to the user's genitals. The genital aperture 106 may be of a variety of shapes and sizes as long as the opening is large enough to allow the genitals to pass through. The posterior of the undergarment covers the user's anus and extends below the user's anus.

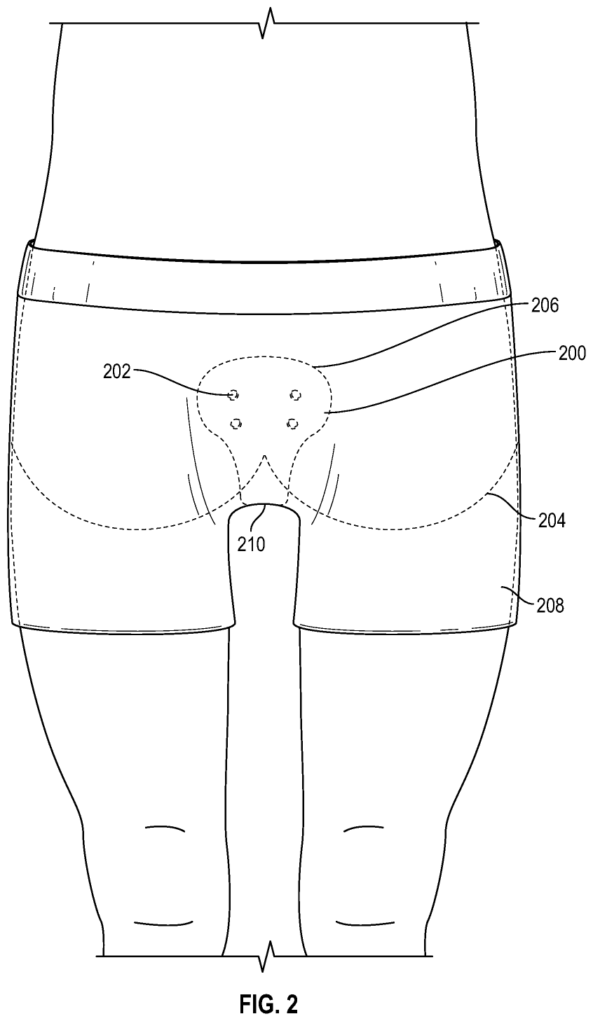

In one embodiment, the incontinence undergarment comprises a front panel 100 extending from the torso aperture to each leg aperture over the user's genitals and, as depicted in FIG. 2, a back panel 208 that covers the user's anus and intergluteal cleft. The front panel 100 and back panel 208 extend downwards until they meet at a point below the user's perineum. The front panel 100 and back panel 208 also extend to the left and right of the user until they meet on the left and right sides of the user. The front panel 100 and back panel 208 are affixed to one another, preferably by stitching the panels together. A genital aperture 106 comprises an oval-like opening on the front panel with the major axis of the opening oriented vertically. The genital aperture 106 is centered with respect to the width of the body and is defined by the boundary 110 of the aperture 106. The highest point of the boundary 110 of the genital aperture 106 is located at a point just above the point where the upper side of the penis meets the body and below the waistband 104 of the undergarment. Preferably, the highest point of the boundary 110 of the genital aperture 106 will be located one to three inches above the point where the upper side of the penis meets the body to avoid pinching the base of the penis as well as allowing clearance for pubic hair.

The lowest point of the boundary 110 of the genital aperture 106 is located at a point just below the point where the bottom of the scrotum meets the body. Preferably, the lowest point of the boundary 110 of the genital aperture 106 will be located 1 to 1.5 inches below the point where the bottom of the scrotum meets the body. The width of the genital aperture 106 can vary as long as sufficient space is provided for the genitals to pass through without causing friction with the undergarment. Preferably, the width of the genital aperture 106 should be between five and seven inches. While this range is applicable to the majority of users, the garment will be offered in different sizes to account for variations in the dimensions of the body.

The undergarment further comprises a nonpermeable patch of material between the user and the inside face of the posterior of the undergarment. The most preferred material for this patch is silicone as silicone is nonpermeable but is also soft and flexible, providing comfort to the user when the undergarment is worn, as well as adjusting to the contours of the user's body as they move. This patch acts as a barrier between the user and the posterior side of the undergarment. If it is to come into contact with stool, the patch area can be much more easily cleaned than if stool should come into contact with other parts of the undergarment. The patch is positioned against the user's body over the anus and is preferably oval in shape, with the major axis of the oval positioned horizontally. The oval shape avoids corners that are more likely to be sensed by a user and create discomfort. This patch extends in width approximately from a midpoint over the left gluteal to a midpoint over the right gluteal, downwards past the anus to a point over the perineum, and upwards to a point below the waistband.

As depicted in FIG. 2, the undergarment may include a nonpermeable removable insert, or guard, 200 between the user and the inside face of the posterior of the undergarment. The guard acts as a barrier between the user and the posterior side of the undergarment. The guard is held in position against the user's body over the anus by the elastic properties of the material of the undergarment.

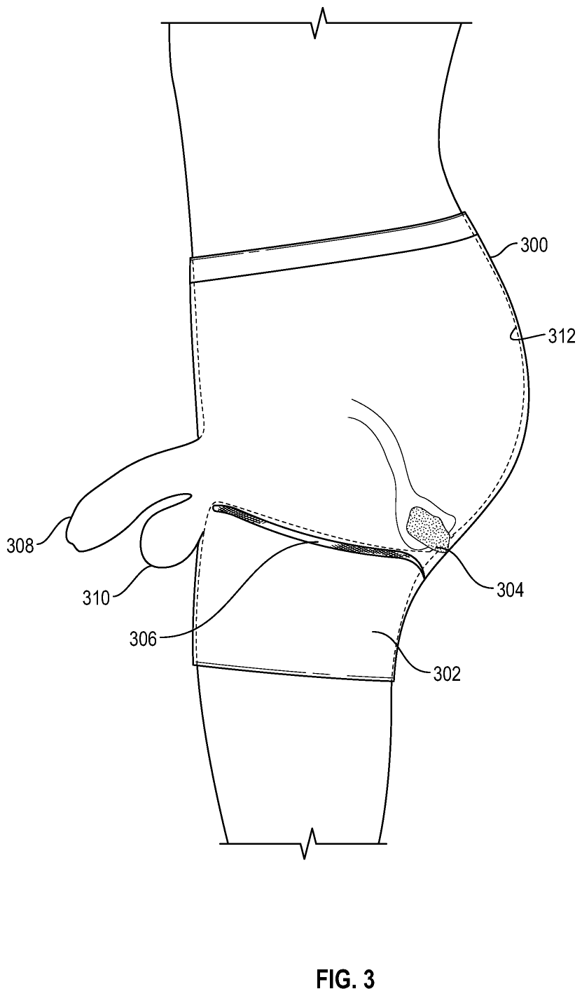

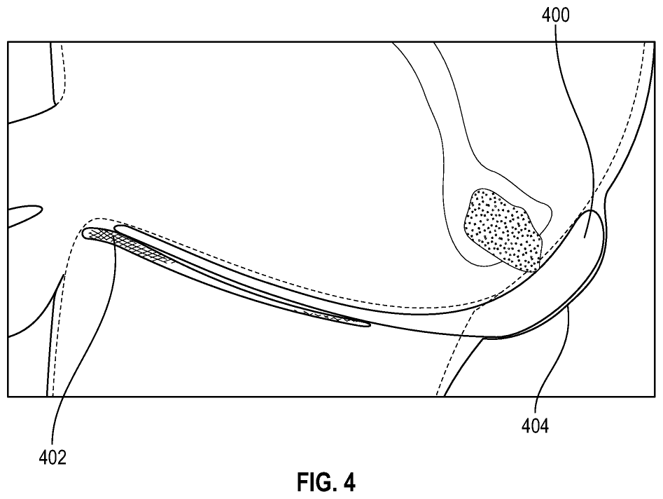

In this embodiment, the leg and torso apertures of the undergarment have a loop of elastic material embedded into the cuffs 102 104 of the openings. This gives the cuffs 102 104 a higher degree of elasticity than the primary material of the garment, which serves several purposes. When the garment is worn, the greater elasticity of the cuffs assists in maintaining the position of the undergarment on the user's body. The elastics around the openings of the undergarment, particularly around the leg and genital openings, also serve as a secondary containment system in case stool should escape beyond the guard. While stool would then soil the fabric of the undergarment, the elastic will prevent stool from escaping beyond the undergarment itself. As shown in FIG. 3, if a bowel movement occurs while the user is wearing the undergarment, the released stool 304 will apply pressure to the posterior panel 300 of the undergarment. By securing the position of the cuffs 102 104 of the leg and torso apertures with embedded elastics, tension will be maintained in the back panel 208 covering the anus and stool 304 is more likely to be contained in the space between the intergluteal cleft and the posterior panel 300 of the undergarment. As shown in FIG. 4, if a guard 400 is used, this embodiment is more likely to keep the guard 400 in place by the same mechanism. Although the cuff 114 of the genital aperture 106 can also contain an elastic, as it is less likely stool may escape from that aperture, it is preferable that the cuff 114 of the genital aperture 106 is stitched and does not contain an elastic.

In another embodiment, there is a strip of fabric 108 306 running from the lower boundary 110 of the genital aperture 106, under the perineum, and towards the rear of the garment, which is thicker than the rest of the garment. The strip of fabric 108 at its thickest point is at least 10 mm, and up to 25 mm thick, the thickness of the strip is defined vertically. The thickness of the strip 108 tapers along a vector that runs parallel to the perineum and in a direction toward the rear of the garment. The strip 108 comprises a stiffer material, such as cotton, regardless of the composition of the remainder of the garment. This thicker material results in a fit where the portion of the garment below the perineum 110 is more tightly in contact with the user's body. This serves as an auxiliary guard to prevent stool from escaping out the genital aperture 106 should the guard 400 fail to contain all of the stool. The strip 108 may be implemented in a number of ways. For example, when used in conjunction with the guard 400, as shown in FIG. 4, the strip 402 may be configured such that the thickest point of the strip 402 is where the strip 108 meets the lower boundary 110 of the genital aperture 106, with the strip 402 tapering towards the rear of the garment. In another example, when used without the guard 400, as shown in FIG. 3, the strip 306 may also be configured such that the thickest point is at a point between the front and rear of the garment and tapers both towards the front and rear of the garment.

In another embodiment, the cuff 114 of the genital aperture 106 also contains an elastic. As this elastic rests against more sensitive areas of the body, this elastic most preferably will be a softer elastic than used in the cuff of the torso aperture, such as a knitted elastic. When an elastic in the genital aperture 106 is used, all apertures of the undergarment contain elastics providing a greater likelihood that the undergarment will be kept in place on the body during physical activity. The elastic around the cuff of the genital aperture 106 also serves as an auxiliary seal in case stool should escape outside of the guard.

The primary material of the undergarment will comprise a blend of materials to achieve a soft and elastic fabric. This ensures that the material will stretch and conform to the user's body while also remaining comfortable against the skin of the user. The material is breathable as users not only generate heat around the areas covered by the undergarment but generate an additional amount of heat during any physical activity, including sexual activity. One type of material that can achieve these characteristics is a blend of 95% cotton and 5% elastane. The tension created by the user's body stretching the elastic material, combined with the rigidity of the cotton, holds the guard 400 securely in place against the user's buttocks surrounding the anus.

Other blends of materials that are elastic, soft, and breathable can also be used. For example, blends, including materials such as rayon, nylon, and polyester, can be used to provide the additional benefit of wicking moisture away from the body. Fabric blends with a higher proportion of stretchable material, such as elastane, may also be used to achieve a tighter fit against the body. For example, in one embodiment, a fabric blend of 90% polyester and 10% elastane may be used. This embodiment would provide greater overall elasticity of the undergarment, which results in both the guard 400 being held more securely against the user's body as well as a greater ability to contain any stool within the undergarment in case stool escapes beyond the guard.

As one of the primary uses of the incontinence undergarment is to contain an involuntary bowel movement during sexual activity, it is possible that the undergarment will come into contact with any number of bodily substances. Although the guard 400 is intended to contain feces if a bowel movement should occur, with repeated use, it is possible that a small amount of stool will come to contact with the fabric of the undergarment itself. Additionally, fluids excreted from the body during sexual activity, as well as perspiration that naturally occurs and is more likely to occur during sexual activity, may also come into contact with the garment. A polyester-based blend provides the further advantage of being more resistant to water-based stains compared to natural fibers. The garment can also be treated with materials such as resin during manufacture to provide further resistance to stains.

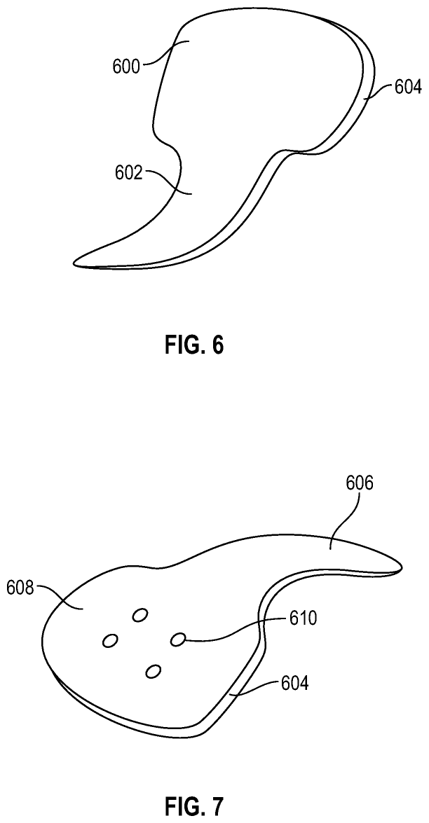

The incontinence undergarment can include a guard 400, which is inserted into the undergarment between the user and the posterior side of the undergarment 300. The guard 400 is comprised of a single body of nonpermeable material such as plastic (including silicone) or rubber. In one embodiment, as shown in FIGS. 6 and 7, the guard is comprised of two sections, an upper portion 600 608 that covers the anus and a lower portion 602 606 that extends along the perineum. The upper portion of the guard 600 608 is centered over the anus and exhibits a slight concave curvature in either direction along the vertical axis on its interior face to conform to the contours of the buttocks. The upper portion of the guard 600 608 also tapers in width both downwards and upwards from its center point over the anus. The upper portion of the guard 600 608 at its widest point is of a width sufficient to extend at least one inch beyond either side of the intergluteal cleft and rest against the left and right buttocks. As the guard extends downwards towards the perineum, it tapers to a much greater degree to lead into the lower portion of the guard 602 606.

The greater tapering towards the bottom of the upper portion of the guard 600 608 results in a narrower lower portion of the guard 602 606. The lower portion of the guard extends from the bottom of the upper portion towards the genital aperture 106. The lower portion of the guard 602 606 terminates at, or very close to, the bottom of the genital aperture 106. The guard does not extend through the genital aperture 106 to the external of the undergarment. The lower portion of the guard 602 606 continues to taper slightly in both height and width as it extends from the bottom of the upper portion of the guard 600 608 to the genital aperture 106. This tapering provides a more comfortable fit by conforming to the narrowing of the intergluteal cleft.

As described above, the strip 108 306 of fabric that runs along the perineum can be configured so that the thickest point of the strip 108 306 is where the strip 108 306 meets the lower boundary of the genital aperture 106, with the strip 108 306 tapering towards the rear of the garment. Thus, the strip 108 306 tapers from the front to the rear of the garment, while the guard tapers in the opposite direction, from the rear to the front of the garment. This allows the combined height of the strip 108 306 and guard 400 to remain relatively consistent along the perineum. This combination provides more comfort towards the front of the garment and a more secure stool-blocking mechanism towards the rear of the garment.

The thickness of the guard 604 should be no more than 25 mm at its thickest point and, preferably, between 5 mm and 15 mm at its thickest point to maintain flexibility. All corners and all edges of the guard will be rounded to provide a smooth surface that is comfortable against the user's skin and minimizes the risk of the guard tearing the fabric of the undergarment. The guard should be constructed from flexible, non-toxic, nonpermeable material such as flexible polyvinyl chloride (PVC), for example. Preferably, flexible PVC made using non-phthalate plasticizers should be used as the guard will be in direct contact with the user's body. Other non-toxic, nonpermeable, flexible materials may also be used, such as silicone, rubber, or other types of plastics which are well-known. The guard may be produced in a variety of colors, including colorless, but most preferably will match the color of the primary material of the undergarment.

In one embodiment of the invention, the elasticity of the undergarment is sufficient to keep the guard in place when the undergarment and guard are worn, as well as during any physical activity that the user may engage in. In another embodiment, the guard has fasteners 610 affixed to its garment-facing side 608. The fasteners can be affixed to the guard in a variety of patterns. For example, the fasteners 610 can be disposed along the perimeter of the guard and will interface with fasteners 202 affixed to corresponding positions on the interior of the undergarment.

The fasteners 202 on the undergarment are arranged in a pattern that corresponds to the fasteners 610 affixed to the guard in order to hold the guard in place. The fasteners 202 on the undergarment should be positioned such that when the guard 400 is secured upon the fasteners 202, the lowest point of the upper portion of the guard 608 is at a point below the anus and above the lowest point of the user's intergluteal fold, and the highest point of the upper portion of the guard 608 is located at a point above the anus and below the waistband 104 of the garment. This ensures that when the fasteners 610 of the guard interface with the fasteners 202 of the undergarment, the guard 400 is in a position that covers the anus but does not extend outside of the undergarment or does not significantly prevent the undergarment from conforming to the user's body. In one embodiment, for example, one set of fasteners may be affixed along a looping path on the inside posterior face of the undergarment 312 that corresponds to the perimeter of the garment-facing side of the upper portion of the guard 608. Another set of fasteners may then be affixed on a linear path on the inside of the portion of the undergarment running under the perineum 110 that corresponds to a linear path along the garment-facing side of the lower portion of the guard 606.

In one embodiment, standard fabric socket and cap snaps commonly found on denim jeans may be used. Socket and cap snaps provide the advantage of consistent positioning of the guard, as then the user will receive audible, visual, and/or physical feedback when each snap is secured. The user is also able to easily disengage the snaps to allow for the removal and reattachment of the guard. In this embodiment, sockets are preferably affixed to the guard and the cap affixed to the undergarment to allow the user to push directly on each cap from the external face of the posterior side of the undergarment to secure it to its corresponding socket. Alternatively, many other fasteners known in the art may be used, such as other types of snaps, hook and loop fasteners, magnets, or pressure-sensitive adhesives.

In another embodiment of the invention, the genital opening 106 is placed lower on the anterior face of the undergarment. This embodiment is more suited for female users as the female genitals are generally located lower on the body and in closer proximity to the anus than on males. In this embodiment, preferably, the highest point of the boundary 110 of the genital aperture 106 is located at a point just above the clitoral hood and below the waistband 104 of the undergarment. Preferably, the highest point of the boundary 110 of the genital aperture 106 will be located one to three inches above the clitoral hood to avoid pinching the sensitive tissue as well as allowing clearance for pubic hair.

The lowest point of the boundary 110 of the genital aperture 106 will be located 1 to 1.5 inches below the lowest point of the labia minora. The width of the genital aperture 106 can vary as long as sufficient space is provided so the labia minora is exposed without causing friction with the undergarment. Preferably, the width of the genital aperture 106 in this embodiment should be between five and seven inches. While this range is applicable to the majority of users, the garment will be offered in different sizes to account for variations in the dimensions of the body.

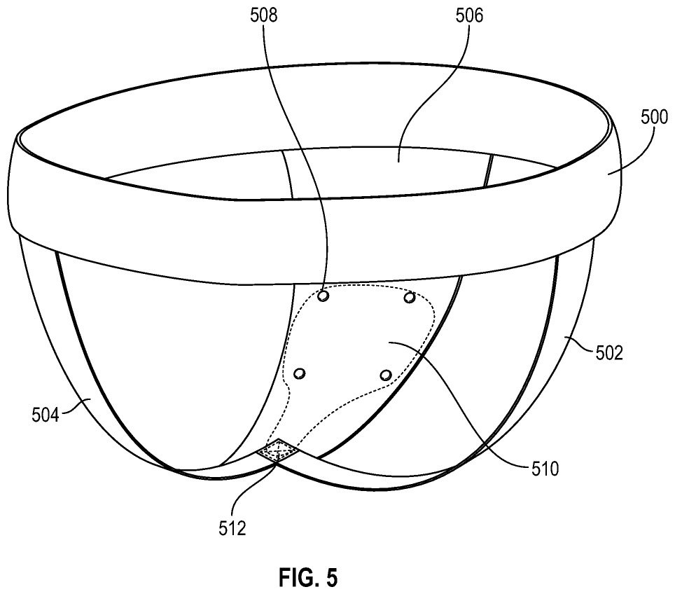

In another embodiment, the undergarment is cut to maximize the size of the apertures for the genital opening and both legs, as depicted in FIG. 5. In this embodiment, the undergarment comprises an elastic waistband 500 similar to the waistband of men's underwear and is also intended to be worn in a manner similar to underwear. A fabric panel 506 is attached to the posterior portion of the waistband 500. When the garment is worn, this panel extends in width approximately from a midpoint over the left gluteal to a midpoint over the right gluteal. This fabric panel 506 extends downwards past the anus to a point over the perineum. The fabric is tapered as it extends downwards, forming a triangular-like shape 506. At a point over the perineum, two fabric straps 502 504 extend outwards from the triangular-like fabric panel 506 and attach to the waistband 500 of the undergarment. One strap 502 attaches to the waistband 500 at approximately the centerline of the left side of the body, while the other strap 504 attaches to the waistband at approximately the centerline of the right side of the body.

In this embodiment, each strap 502 504 rests against the body in a diagonal position, extending point over the perineum upwards and distally to the waistband 500. Together with the waistband, the straps form the perimeter of the genital aperture. The straps 502 504, waistband 500, and posterior fabric panel 506 form the perimeter of each leg aperture. As is the case with other embodiments, an elastic loop is contained within the waistband 500. As the straps 502 504 in this embodiment form the cuffs or perimeter of both the leg and genital openings, an elastic may or not be included within the straps.

This configuration provides sufficient fabric over the anus and intergluteal cleft to entirely cover the guard 400 as well as to hold the guard 400 in place while providing greater apertures for the genitals and for each leg. This particular configuration may be preferable in cases where the user generates a significant amount of heat, as can be common during sexual activity. This configuration may be preferable if the user wishes to wear a second undergarment over the incontinence undergarment until access to the genitals is desired. This configuration may also be preferred by women as the diagonal straps present a style more similar to women's underwear. Additionally, the minimal fabric allows for greater accessibility to a woman's genitals due to the shorter distance between the anus and female genitals.

In this embodiment, the two fabric straps 502 504 that connect to the triangular fabric panel 506 can be stitched 512 to the triangular fabric like the other straps of the undergarment are. In another variation, the ends of the two fabric straps that connect to the triangular panel are stitched to each other but connect to the triangular panel using an adjustable fastener. For example, hook and loop fasteners can be used with a hook panel affixed to the outside face of the front strap and a loop panel affixed to the inside face of the back strap. This configuration permits the user to adjust the distance between the lower boundary 110 of the genital opening and the lower point of the guard. As discussed earlier, this is particularly advantageous for female users due to the shorter distance between the anus and female genitals. The adjustable point of contact between the two fabric straps 502 504 forming the genital opening and the triangular fabric 506 panel allows for a customized fit to ensure the genitals are not covered by the garment while the anus is still covered by the guard 400.

The guard used in this embodiment is shorter in length, given the reduced material towards the front of the garment. In this embodiment, the lower portion of the guard 602 606 is substantially shorter in length, extending to the point where the two fabric straps connect to the triangular fabric panel 512. A reduced number of fasteners 508, or a single fastener, are placed on the portion of the garment that interfaces with the lower portion of the guard 606. This embodiment of the guard provides protection for containing stool while also limiting the extent to which the guard extends beyond the garment and is visible from the outside.

In another embodiment, the guard comprises a non-reusable, disposable barrier that blocks stool within the intergluteal cleft. This provides the advantage of not having to clean the guard after it has come into contact with stool and instead to simply replace it. A disposable guard comprises a barrier that can be constructed from a variety of absorbing materials that are known in the art, such as cotton, superabsorbent polymers, or wood pulp. The garment-facing side 606 608 of the disposable guard comprises a layer of nonpermeable film such as polyethylene or polypropylene. These materials are well-known in the art and are commonly used in diapers and sanitary napkins.

The guard in this embodiment effectively traps stool, preventing it from contaminating the remainder of the undergarment while also being able to be produced at a low cost. This makes it economical for the guard to be disposed of after each use rather than having to clean it for reuse. The garment-facing side 606 608 of the disposable guard is also affixed with fasteners 202 in a manner similar to the non-disposable guard as described above. As one of the features of the disposable guard is its low cost, the fasteners 202 preferably used by the disposable guard comprise hook and loop fasteners or pressure-sensitive adhesives such as those commonly used in women's sanitary napkins.

The fabric of the undergarment may also be treated with materials that minimize or control odors. As described above, due to the nature of the primary use case for the product, feces, perspiration, and other bodily fluids normally excreted from the body during sexual activity may all come into contact with the undergarment. The bacteria contained in these substances can create unpleasant odors. Treating the fabric of the undergarment with materials that destroy these bacteria can mitigate this issue. A variety of compounds known in the art can be used to treat the fabric of the undergarment, including antibacterial compounds (e.g., triclosan or triclocarban), silver particles, zeolites, or hydrogen peroxide.

An object of the invention is to provide a mechanism for containing involuntary bowel movements during physical activities, which can provide the user with peace of mind and restore his confidence by limiting the possibility of unpleasant accidents during those activities. In the context of sexual activity, this can be critical as sexual dysfunction, such as erectile dysfunction, can be triggered by anxiety. Minimizing anxiety can not only improve the physical mechanisms of the body involved in sex but also improve the dynamic between partners.

The invention can be used in conjunction with medications, such as those used to treat erectile dysfunction (e.g., sildenafil) or those used to treat incontinence (e.g., hyoscyamine). In some cases, users may achieve optimal results by initially using the invention in conjunction with such medications until they become accustomed to wearing the invention during sex. Users can then taper off medications and engage in sexual activities wearing the invention without having to also rely on medications. In other cases, users will find that medications used in conjunction with the invention continuously will achieve better results for them. It should be noted that the invention will work most effectively with users that have healthy stool that does not contain an excessive amount of water. The effectiveness of the invention in containing stool may be diminished with an increased amount of water in the stool.

Unlike traditional incontinence devices that rely on bulky absorption materials, the invention is designed to be aesthetically pleasing and comfortable to detract minimally from sexual activities. As a result, the undergarment can also be used for fetish or kink purposes, regardless of the user's incontinence. Unlike medications, enemas, or other approaches to handling incontinence during sex, an object of the invention is to provide a solution to participating in sexual activity for incontinent individuals that requires minimal planning and can be put into use on short notice. This permits more spontaneity, which can also improve a user's sexual performance and sexual dynamic with their partner.

The invention can also be used without the guard for a user who is not incontinent. It is common in some males to generate excessive amounts of perspiration around the genitals due to genetics, weather, activity, or medical conditions. When the male's genitals, in particular the scrotum, come into contact with other parts of the body, such as the inner thigh or the inguinal canal, this perspiration can cause sticking, which generates discomfort for the individual. The genital opening provided by the invention serves as a barrier of fabric between the person's genitals and the rest of their body, preventing such sticking and relieving such discomfort for the user.

The detailed embodiments of the present invention disclosed herein are merely exemplary of the invention that may be embodied in various and alternative forms. The figures are not necessarily to scale, and some features may be exaggerated or minimized to show details of particular embodiments, features, or elements. Specific structural and functional details, dimensions, or shapes disclosed herein are not limiting but serve as a basis for the claims and for teaching a person of ordinary skill in the art the described and claimed features of embodiments of the present invention. Many variations, combinations, modifications, or equivalents may be substituted for elements thereof without departing from the scope of the invention.

The above-detailed description is merely exemplary in nature and is not intended to limit the described embodiments or the application and uses of the described embodiments. Any implementation or embodiment described herein as “exemplary,” “illustrative,” or “representative” is not necessarily to be construed as preferred or advantageous over other implementations. All of the implementations described above are exemplary implementations provided to enable persons skilled in the art to make or use the embodiments of the disclosure and are not intended to limit the scope of the disclosure, which is defined by the claims.

Furthermore, there is no intention to be bound by any expressed or implied theory presented in the preceding technical field, background, brief summary, or detailed description. It is also to be understood that the specific devices and processes illustrated in the attached drawings, and described in the specification, are simply exemplary embodiments of the inventive concepts defined in the appended claims. Hence, specific dimensions and other physical characteristics relating to the embodiments disclosed herein are not to be considered limiting unless the claims expressly state otherwise.

What is claimed is:

1. A fecal incontinence undergarment comprising:
an elastic waistband defining a torso aperture for a user's torso;
elastic loops with greater elasticity than said elastic waistband defining leg apertures for each of the user's legs;
a front panel extending from the torso aperture to each leg aperture, the front panel containing a genital aperture positioned over the user's genitals which allows the user's genitals to pass through to the exterior of the undergarment;
a back panel covering the user's anus and intergluteal cleft;
wherein the undergarment is configured to be worn by the user like underwear.

2. The undergarment of claim 1,
further comprising a patch on the interior of the undergarment that is flexible and nonpermeable, spanning in width from a midpoint of the user's left buttock to a midpoint of the user's right buttock, spanning in length from a point above the user's anus and below the highest point of the user's intergluteal cleft to a point below the anus, tapering in width towards the bottom of the patch, wherein the patch is positioned over the user's anus.

3. The undergarment of claim 1, further comprising:
a guard that is removable, flexible, nonpermeable, devoid of sharp corners or edges, spanning in width from a midpoint of the user's left buttock to a midpoint of the user's right buttock, spanning in length from a point above the user's anus and below the highest point of the user's intergluteal cleft to a point below the anus, tapering in width towards the bottom of the guard, wherein the guard is positioned over the user's anus between the user and the interior of the undergarment.

4. The undergarment of claim 3, further comprising:
fasteners on the garment-facing side of the guard and corresponding fasteners on the interior face of the back panel of the undergarment, wherein the fasteners on the guard are capable of interfacing with the fasteners on the garment to form a physical connection.

5. The undergarment of claim 1, further comprising:
a strip of fabric affixed to the interior of the garment, under the user's perineum, between the front panel and back panel of the garment.

6. The undergarment of claim 3, further comprising:
a strip of fabric affixed to the interior of the garment, under the user's perineum, between the front panel and back panel of the garment.

7. The undergarment of claim 4, further comprising:
a strip of fabric affixed to the interior of the garment, under the user's perineum, between the front panel and back panel of the garment, wherein the strip of fabric has a thickness between 10 mm and 25 mm.

8. An fecal incontinence undergarment comprising:
an elastic waistband defining a torso aperture for a user's torso;
a three-sided back panel covering the user's anus and intergluteal cleft, spanning in length from the waistband of the undergarment to a point under the user's perineum, the top of the back panel spanning in width from a point over the user's left buttock to a point over the user's right buttock, tapering in width as it extends downwards until two sides of the back panel meet at a point at the bottom of the panel;
two leg straps extending from the point at the bottom of the back panel to discrete points on the front waistband, defining a genital aperture that allows the user's genitals to pass through to the exterior of the undergarment;
wherein the undergarment is configured to be worn by the user like underwear.

9. The undergarment of claim 8, further comprising:
a patch on the interior of the back panel that is flexible and nonpermeable, spanning in length from a point above the user's anus and below the highest point of the user's intergluteal cleft to a point below the anus, contained within the perimeter of the back panel, tapering in width towards the bottom of the patch, wherein the patch is positioned over the user's anus.

10. The undergarment of claim 9, further comprising:
a guard that is removable, flexible, nonpermeable, devoid of sharp corners or edges, spanning in width from a midpoint of the left buttock to a midpoint of the right buttock, spanning in length from a point above the user's anus and below the highest point of the user's intergluteal cleft to a point below the anus and above the lowest point of the user's intergluteal fold, tapering in width towards the bottom of the guard;
wherein the guard is positioned over the user's anus between the user's buttocks and the interior face of the back panel of the undergarment and does not extend beyond the perimeter of the back panel.

11. The undergarment of claim 10, further wherein:
the three-sided back panel connects to the two leg straps using an adjustable fastener, permitting the distance between the lower boundary of the genital opening and the lower point of the guard to be adjusted.

12. The undergarment of claim 10, further comprising:
fasteners on the garment-facing side of the guard and corresponding fasteners on the interior face of the back panel of the undergarment,
wherein the fasteners on the guard are capable of interfacing with the fasteners on the garment to form a physical connection.

13. The undergarment of claim 12, further wherein:
the three-sided back panel connects to the two leg straps using an adjustable fastener, permitting the distance between the lower boundary of the genital opening and the lower point of the guard to be adjusted.

14. A method of containing bowel movements during a user's sexual activity comprising the steps of:
obtaining an undergarment comprising a back panel positioned over the anus to contain any stool released within the intergluteal cleft and a front panel containing a genital aperture positioned over the user's genitals, which allows the user's genitals to pass through to the exterior of the undergarment;
wearing the undergarment at some point prior to engaging in sexual activity;
engaging in sexual activity;
removing the undergarment following sexual activity; and
cleaning or disposing of the undergarment if a bowel movement occurred during sexual activity and fecal matter made contact with the undergarment.

15. The method of claim 14, further comprising the steps of:
obtaining a guard that is removable, flexible, nonpermeable, devoid of sharp corners or edges, the guard having an anterior face with a surface area that is sufficient to cover the user's anus but less than the surface area of the interior face of the back panel;
positioning the guard over the user's anus between the user's buttocks and the interior face of the back panel of the undergarment;
removing the guard from the undergarment following sexual activity; and
cleaning or disposing of the guard if a bowel movement occurred during sexual activity.

16. The method of claim 14, further comprising the steps of:
obtaining an undergarment with fasteners on the garment-facing side of the guard and corresponding fasteners on the interior face of the back panel of the undergarment,
engaging the fasteners on the guard with the fasteners on the garment to form a physical connection that positions the guard over the user's anus; and
disengaging the fasteners on the guard with the fasteners on the garment when removing the guard.

17. The undergarment of claim 14, further comprising the steps of:
obtaining an undergarment with a strip of fabric affixed to the interior of the garment, under the user's perineum, between the front panel and back panel of the garment.

18. The undergarment of claim 15, further comprising the steps of:
obtaining an undergarment with a strip of fabric affixed to the interior of the garment, under the user's perineum, between the front panel and back panel of the garment.

19. The undergarment of claim 16, further comprising the steps of:
obtaining an undergarment with a strip of fabric affixed to the interior of the garment, under the user's perineum, between the front panel and back panel of the garment.
